(19) United States
(12) Patent Application Publication
(10) Pub. No.: US 2011/0270090 A1
(43) Pub. Date: Nov. 3, 2011

(54) NEEDLE HAVING ULTRASOUND OPAQUE ELEMENTS

(76) Inventors: Timothy Earl Morey, Gainesville, FL (US); Francesca Kayser Enneking, Gainesville, FL (US)

(21) Appl. No.: 13/003,966

(22) PCT Filed: Sep. 9, 2009
(86) PCT No.: PCT/US2009/056324

§ 371 Date: Feb. 24, 2011

Related U.S. Application Data
(60) Provisional application No. 61/098,025, filed on Sep. 18, 2008.

(51) Int. Cl. A61B 8/00; B23P 11/00; A61M 5/00

(52) U.S. Cl. 600/461; 604/272; 29/428

(57) ABSTRACT

Embodiments of the present invention are related to techniques for increasing the visibility of needles while using ultrasound or other imaging technologies. For example, one embodiment of the present invention is directed to a needle that includes a longitudinal needle-shaped base structure and at least one enclosure positioned circumferentially about the base structure. The needle also includes at least one core defined in the at least one enclosure, wherein the at least one core comprises a gas and/or fluid that is configured to limit propagation of ultrasonic energy therethrough.

### BACKGROUND

Ultrasound is a common technique used to visualize a needle while using the needle to perform various medical procedures such as injecting drugs, aspirating tissue, positioning catheters within a patient's body, or performing a biopsy. For instance, visualization of the needle becomes very important for regional anesthesia, especially in order to avoid damaging structures like organs, nerves, and vessels. Visualization of the needle also aids in providing the requisite amount of anesthesia for a successful nerve block and avoiding complications such as intraneural or intravascular injection. Different techniques have been employed to enhance the visibility of the needle using ultrasound. For example, forming dimples or other disruptions on the surface of the needle have been used to improve the “echogenicity” of the device. Despite these improvements in visibility, there is a need for systems and methods for increasing the visibility of needles and other devices within a patient's body while using ultrasound or other imaging technologies.

### BRIEF DESCRIPTION OF THE DRAWINGS

Having thus described the invention in general terms, reference will now be made to the accompanying drawings, which are not necessarily drawn to scale, and wherein:

FIG. 1 shows a needle assembly according to one embodiment of the invention.

FIG. 1A illustrates an enlarged and cross-sectional view of a core according to an embodiment of the present invention.

FIG. 2 depicts an enlarged view of the needle assembly shown in FIG. 1.

FIG. 3 illustrates a cross-sectional view of a needle according to one embodiment of the present invention.

### DETAILED DESCRIPTION OF VARIOUS EMBODIMENTS OF THE INVENTION

Various embodiments of the present invention will now be described more fully hereinafter with reference to the accompanying drawings, in which various embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

### Overview

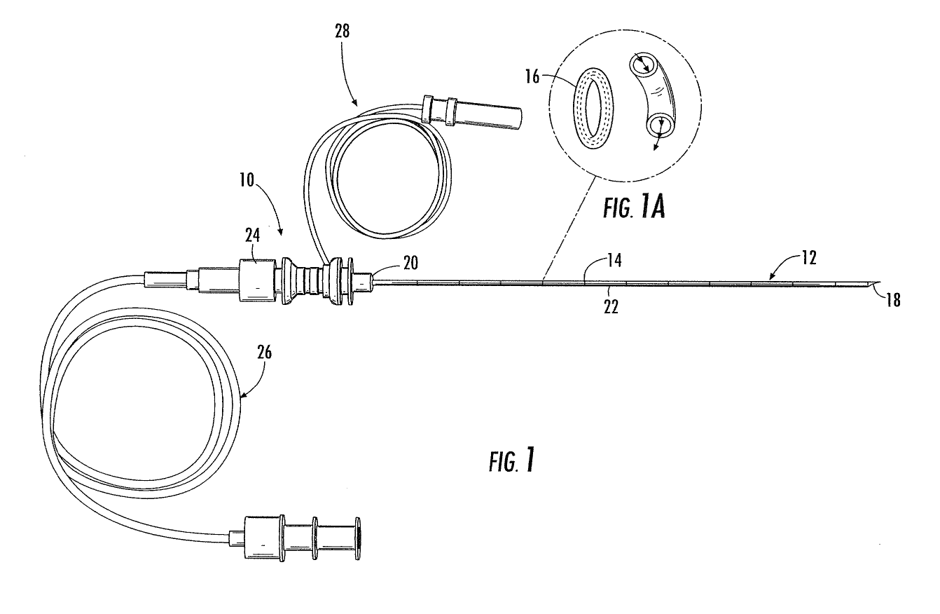

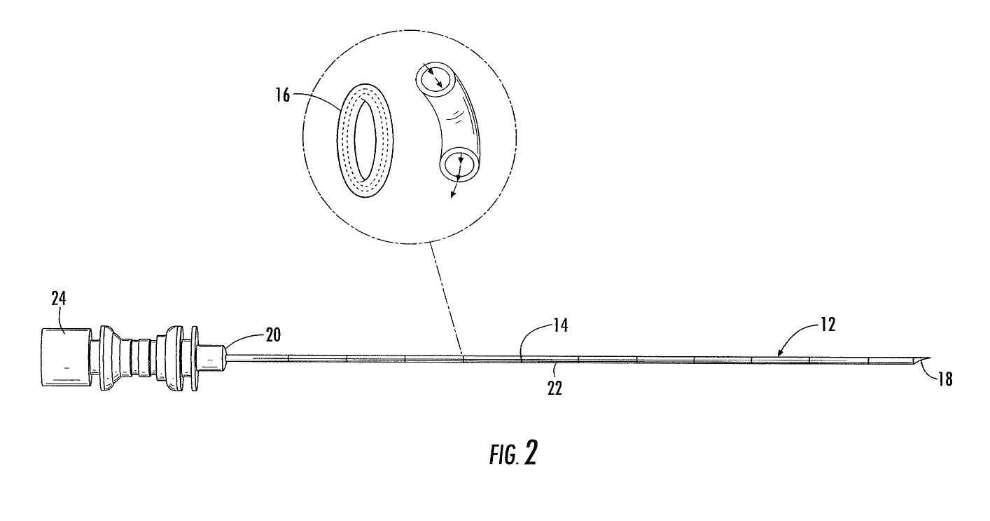

Various embodiments of the present invention are configured to provide techniques for increasing the visualization of a needle in conjunction with ultrasound. FIGS. 1 and 2 depict an exemplary embodiment of a needle assembly 10. The needle assembly 10 includes a needle 12, a plurality of gas and/or fluid enclosures 14 located along the length of the needle, and a plurality of circumferential cores 16 defined within the respective enclosures. According to one embodiment, the cores 16 are filled with a gas and/or fluid to enhance the echogenicity of the needle 10.

Needle

According to one embodiment of the present invention, the needle assembly 10 comprises a needle 12 that, in various embodiments, includes a pointed or beveled tip 18 at its distal end to facilitate penetration through tissue. The needle 12 may alternatively include a blunt or rounded tip, such as for epidural or paravertebral blocks. Moreover, the needle 12 may further include a proximal end 20 coupled to a handle or hub 24, and a longitudinal lumen 22 extending between the proximal and distal ends of the needle. The hub 24 may include one or more connectors configured to be coupled with tubes 26, 28 for removing or administering fluid and/or tissue through the lumen of the needle 12, facilitating placement of catheters, or conveying an electrical current. The needle 12 may be comprised of any of various materials, such as a semi-flexible polymeric material (e.g., PTFE) that is biocompatible and capable of penetrating tissue without kinking but also capable of conforming to various contours within a patient's body.

In various embodiments, the needle 12 may be used in various medical procedures, such as drug delivery, aspiration of tissue and fluids, placement of catheters and wires, and/or biopsy procedures. For example, the needle 12 may be used for regional anesthesia placement, injections for chronic pain, injection of intraocular drugs, injection of chemotherapeutic agents into tumors, aspirating tissue from masses including tumors arising from various structures as well as fetal tissue (e.g., aminiocentesis, chroiocentesis, twin-twin, intrauterine blood transfusions), placement of vascular catheters, placement of wires guides (such as for intraveneous or intraarterial catheter placement) placement of peripherally inserted central catheters, placement of central venous catheters, placement of catheters to aspirate air or fluids, placement of pulmonary artery catheters, and placement of cardiac catheterization catheters.

Grooves

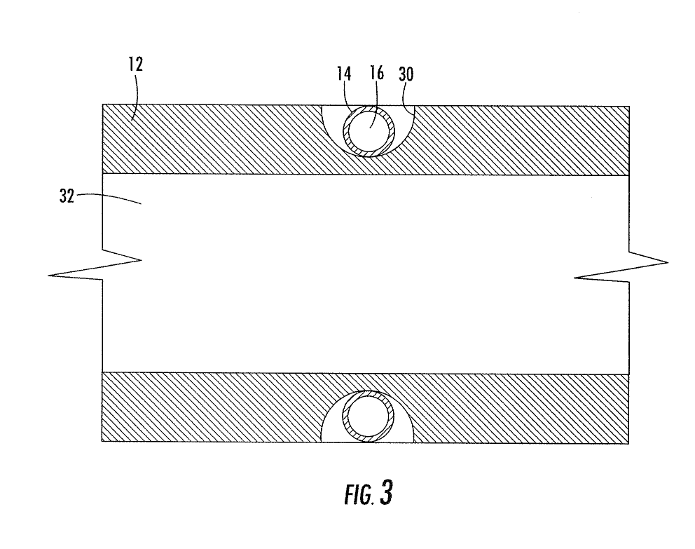

According to one embodiment, the needle 12 may include a plurality of grooves defined within its outer surface. The grooves 30 could be defined in the needle 12 such that the grooves do no extend into the lumen 32 of the needle, as shown in FIG. 3. For instance, the grooves 30 may be concave in configuration and extend partially within the thickness of the wall of the needle 12 and circumferentially about the needle. Each groove 30 may be configured to receive a respective gas and/or fluid enclosure 14 at least partially therein, as described in further detail below. The grooves 30 defined in the needle 12 may be formed using various techniques, such as cutting within the needle or molding the enclosures in the needle when forming the needle. Although the grooves 30 may be concavely curved to conform to the curvature of the enclosures 14, the grooves need not be concavely curved, as the grooves may be V-shaped or rectangular in cross-section as long as the grooves are sized and configured to at least partially receive an enclosure therein. Although the grooves 30 are shown as being defined circumferentially about the needle 12, the grooves could be defined axially along the needle, such as in predetermined lengths defined along the longitudinal axis of the needle.

Gas/Fluid Enclosures

According to one embodiment of the present invention, a plurality of gas and/or fluid enclosures 14 are located between the proximal end 20 and the tip 18 of the needle 12 and about the circumference of the needle. The enclosures 14 may be tubular enclosures or hollow rings that are configured to extend about the circumference of the needle 12. Thus, the enclosures 14 may include a convex outer surface that is configured to conform to the concave grooves 30, as shown in FIG. 3. Any number of enclosures 14 may be used and if more than one enclosure is employed, the enclosures may be spaced in predetermined or equidistant intervals along the length of the needle 12. For instance, the enclosures 14 may be spaced apart from one another by about 1 cm, as shown in FIGS. 1 and 2. In addition, one enclosure 14 may be located proximate to the tip 18 of the needle 12.

The number, size, and configuration of the enclosures 14 may vary and, as explained in further detail below, in various embodiments, each enclosure may include a respective core 16 for receiving a gas and/or fluid therein. For example, the enclosures 14 may have a radius of about 0.10 to 2 mm for a needle having a radius of about 0.5 to 3 mm. In addition, although the enclosures 14 are preferably located about the entire circumference of the needle 12 such that visualization is not affected by the orientation of the needle, the enclosures could be located partially about the circumference of the needle (e.g., halfway about the circumference), or different enclosures could be defined at different circumferential locations about the needle. If desired, the enclosures 14 could be disposed axially along a longitudinal axis of the needle 12 rather than circumferentially about the needle. Furthermore, the enclosures 14 may also be various materials, such as a biocompatible polymeric material.

According to one embodiment, the enclosures 14 may be positioned within the tubular wall of the needle 12 such that the enclosures do not extend beyond the outer surface of the needle, as shown in FIG. 3. Thus, the enclosures 14 may be positioned within grooves 30 such that the enclosures are below or flush to the outer surface of the needle 12. As such, in various embodiments, the enclosures 14 may not interfere with fluid and/or tissue traveling through the lumen 32 of the needle 12. However, it is understood that the enclosures 14 could be attached to the outer surface of the needle 12 rather than being positioned within grooves 30, extend partially within the wall of the needle such that the enclosures extend beyond the outer surface of the needle, or extend partially within the lumen of the needle. For example, the enclosures 14 may be coupled to the needle 12 using various techniques such with an adhesive, microwelding, or a press fit. Furthermore, the enclosures 14 may be integrally formed in the needle 12 in order to reduce the potential for infection, which could result from the enclosures breaking off or otherwise being left in the patient's body.

Circumferential Core

In various embodiments, the needle assembly 12 includes a plurality of circumferential cores 16 defined within respective enclosures 14. The cores 16 may be a gas and/or fluid that appears opaque or has a different echogenicity signal than surrounding tissue when imaged using ultrasound. For instance, the cores 16 may be filled with oxygen, carbon dioxide, hydrogen, helium, nitrogen, air, sulphur, hexafluoride, argon, or xenon gas. Exemplary fluid cores 16 include saline, water, or lactate ringers. The cores 16 could also be a combination of gas and fluid. The gas and fluid mixture could be agitated before insertion so that microbubbles could be viewed using ultrasound. As such, the enclosures 14 may be hollow to define the cores 16, as shown in FIG. 1A. Thus, the cores 16 may be a tubular ring of gas and/or fluid defined within the enclosures 14 and that also extend about the entire circumference of the needle 12. However, the cross section of the cores 16 need not necessarily be circular, and the cores could be defined partially about the circumference of the enclosures or could be defined at different circumferential locations within the enclosures. Moreover, the number, size, and configuration of the enclosures 14 may vary such that number, size, and configuration of the cores 16 may also vary.

In various embodiments, the particular gas and/or fluid used is capable of limiting propagation of ultrasonic energy therethrough. The gas and/or fluid core 16 may be injected within the enclosures 14, or the enclosures may entrap a gas and/or fluid therein to form the cores 16, such as with injection molding. Therefore, the cores 16 may enhance the echogenicity of the needle 12 when using ultrasound since the gas and/or fluid will appear opaque when imaging the needle using ultrasound. In particular, the cores 16 may allow the physician to visualize the distal tip 18 of the needle 12, as well as the location of the needle within the body using the spaced cores along the length of the needle.

### Exemplary Method

In particular embodiments, a needle assembly 10 is employed to perform a medical procedure. Generally, the method includes delivering the needle 12 into a patient's body such that at least one of the enclosures 14 is positioned within the patient's body. Typically, the physician urges the tip 19 of the needle 12 through the patient's skin and to a particular area of interest. In various embodiments, at least one enclosure 14 is positioned proximate to the distal end of the needle 12 so that the tip may be readily located by applying ultrasound to the cores 16 using ultrasound imaging techniques. As the needle 12 is inserted further within the body, the physician can monitor the location of the depth of the needle within the body. For instance, the depth of the needle 12 may be measured by: (1) determining the number of enclosures 14 remaining outside the patient's body, which allows the physician to determine the number of enclosures within the body based on a predetermined spacing between the enclosures (e.g., the enclosures could be different colors to aid the physician in determining the depth outside of the patient's body); or (2) determining the number of cores 16 visualized within the body using ultrasound imaging (e.g., if 3 rings are visualized within the patient, a physician can determine the depth by multiplying 3 times the predetermined spacing between the cores).

Ultrasonic energy is applied to the patient's body proximate to the needle 12 using any desired ultrasonic imaging technique known to those of ordinary skill in the art. Thus, the needle assembly 10 may be used with any number of medical procedures using ultrasound. Moreover, the patient's body may be imaged in cooperation with the application of ultrasound using imaging devices known to those of ordinary skill in the art, such that the gas and/or fluid within each of the cores 16 may limit propagation of ultrasonic energy therethrough and appears opaque. For example, the physician may be able to visualize the location of the needle 12 using a video monitor in real time. Thus, the physician is able to readily see the cores 16 when imaging the patient's body so as to determine the location of the needle 12.

According to one embodiment, the needle 12 may be employed for placement of brachial plexus anesthesia using the Infraclavicular approach for open fixation of a fractured radius. In this case, the needle 12 is directed towards the brachial plexus and may be vaguely visualized with the ultrasound probe placed on the patient's skin. Thereafter, some anesthetic may be injected, and the anesthetist may look for the injection of this fluid using ultrasound and subsequently move the needle 12 to the correct location where additional anesthetic may be injected. By visualizing the needle 12 with ultrasound, the anesthetist is able to accurately and efficiently maneuver the needle within the body and to the desired location, while reducing the incidence of complications.

### CONCLUSION

It is understood that although the enclosures 14 and cores 16 have been described in the context of performing a medical procedure with needles, such use is not meant to be limiting, as similar techniques may be used with various medical devices, such as a tube, a catheter, cannula, or the like, for use with ultrasound or other imaging techniques (e.g., x-ray or CT scans) in any desired area of the body. Embodiments of the present invention may facilitate the determination of the exact location of the needle tip clearly to reduce the time of block placement (improved health care expenses), frequency of success, and reduce complications (e.g., intraneural or intravascular injection) to improve patient safety.

Many modifications and other embodiments of the invention will come to mind to one skilled in the art to which this invention pertains having the benefit of the teachings presented in the foregoing descriptions and the associated drawings. For example, as will be understood by one skilled in the relevant field in light of this disclosure, the invention may take form in a variety of different mechanical and operational configurations. Therefore, it is to be understood that the invention is not to be limited to the specific embodiments disclosed and that modifications and other embodiments are intended to be included within the scope of the appended exemplary concepts. Although specific terms are employed herein, they are used in a generic and descriptive sense only and not for the purposes of limitation.

That which is claimed:

1. A needle comprising:
a longitudinal needle-shaped base structure;
at least one enclosure positioned circumferentially about the base structure; and
at least one core defined in the at least one enclosure, wherein the at least one core comprises a gas and/or fluid that is configured to limit propagation of ultrasonic energy therethrough.

2. The needle of claim 1, further comprising a plurality of enclosures each positioned circumferentially about the needle and spaced apart from one another, and a plurality of cores defined in a respective one of the plurality of enclosures.

3. The needle of claim 2, wherein the needle comprises proximal and distal ends, and wherein the plurality of enclosures are equidistantly spaced from one another between the proximal and distal ends.

4. The needle of claim 1, further comprising at least one groove defined in an outer surface of the needle-shaped base structure.

5. The needle of claim 4, wherein the at least one enclosure is configured to be at least partially positioned within the at least one groove.

6. The needle of claim 5, wherein the at least one enclosure is positioned within the at least one groove such that the enclosure is flush with or below the outer surface of the base structure.

7. The needle of claim 4, wherein the at least one groove defines a concave surface within the base structure.

8. The needle of claim 7, wherein the at least one enclosure defines a convex surface that is configured to at least partially conform to the concave surface defined by the at least one groove.

9. The needle of claim 1, wherein the at least one enclosure comprises a hollow ring, and wherein the core comprises a gas and/or fluid entrapped within the hollow ring.

10. The needle of claim 1, wherein the at least one enclosure extends completely about a circumference of the base structure.

11. The needle of claim 1, wherein the at least one core extends completely about a circumference of the base structure.

12. The needle of claim 1, wherein the at least one core is selected from a group consisting of: oxygen, carbon dioxide, hydrogen, helium, nitrogen, air, sulphur, hexafluoride, argon, xenon gas, saline, water, lactate ringers, and combinations thereof.

13. A method of imaging a patient's body comprising:
delivering a longitudinal needle within a patient's body, the needle comprising at least one gas and/or fluid core defined within at least one enclosure, wherein the enclosure is positioned circumferentially about the needle;
applying ultrasonic energy to the patient's body; and
imaging the patient's body such that the at least one gas and/or fluid core limits propagation of ultrasonic energy therethrough and appears opaque.

14. The method of claim 13, wherein the needle is configured to perform a procedure selected from a group consisting of: drug delivery, aspiration of tissue and fluid, placement of a catheter or wire, biopsy, and combinations thereof.

15. A method of forming a needle comprising:
defining at least one groove circumferentially about a longitudinal needle;
positioning at least one enclosure at least partially within the at least one groove, wherein the at least one enclosure comprises a gas and/or fluid core that is configured to limit propagation of ultrasonic energy therethrough.

16. The method of claim 15, wherein defining comprises defining a plurality of grooves circumferentially about the needle and spaced apart from one another, and wherein positioning comprises positioning a plurality of enclosures in a respective one of the plurality of grooves.

17. The method of claim 15, wherein defining comprises defining at least one groove within an outer surface of the needle.

18. The method of claim 17, wherein defining comprises defining at least one concave groove within the outer surface of the needle.

19. The method of claim 18, wherein the at least on enclosure comprises a convex surface, and wherein positioning comprises positioning the at least one enclosure within the groove such that the convex surface at least partially conforms to the concave surface defined by the at least one groove.

20. The method of claim 17, wherein positioning comprises positioning the at least one enclosure within the at least one groove such that the enclosure is flush with or below the outer surface of the needle.

21. The method of claim 15, wherein defining comprises defining the at least one groove completely about a circumference of the needle.
